(19) United States
(12) Patent Application Publication
(10) Pub. No.: US 2022/0282249 A1
(43) Pub. Date: Sep. 8, 2022

(54) COMPOUNDS AND METHODS USEFUL FOR MODULATING GENE SPLICING

(71) Applicant: ARNAY SCIENCES, LLC, Shrewsbury, MA (US)

(72) Inventor: Sudhir Agrawal, Shrewsbury, MA (US)

(21) Appl. No.: 17/697,081

(22) Filed: Mar. 17, 2022

Related U.S. Application Data
(63) Continuation of application No. PCT/US2020/023598, filed on Mar. 19, 2020.
(60) Provisional application No. 62/902,603, filed on Sep. 19, 2019.
(60) Provisional application No. 62/943,539, filed on Dec. 4, 2019.

(51) Int. Cl. C12N 15/113; A61K 31/7088; C12N 15/11

(52) U.S. Cl. CPC C12N 15/113; A61K 31/7088; C12N 15/111; C12N 2310/11; C12N 2310/315; C12N 2310/321; C12N 2320/33; C12N 2310/346; C12N 2310/3231

(57) ABSTRACT

The present invention is directed to compounds, compositions, and methods useful for modulating gene splicing. In some embodiments, modulating gene splicing increases expression of a target protein or a target functional RNA.

### RELATED APPLICATIONS

This application is a continuation of International Application No. PCT/US2020/023598, which designated the United States and was filed on Mar. 19, 2020, published in English, which claims the benefit of U.S. Provisional Application No. 62/902,603, filed on Sep. 19, 2019 and U.S. Provisional Application No. 62/943,539, filed on Dec. 4, 2019. The entire teachings of the above applications are incorporated herein by reference.

### BACKGROUND

The potential for the development of an antisense oligonucleotide therapeutic approach was first suggested in articles published 1978. Zamecnik and Stephenson, Proc. Natl. Acad. Sci. U.S.A. 75: 280-284 and 285-288 (1978); discloses that a 13-mer synthetic oligonucleotide that is complementary to a part of the Rous sarcoma virus (RSV) genome inhibits RSV replication in infected chicken fibroblasts and inhibits RSV-mediated transformation of primary chick fibroblasts into malignant sarcoma cells.

An antisense oligonucleotide approach makes use of sequence-specific binding of DNA and/or RNA based oligonucleotides to selected mRNA, microRNA, preRNA or mitochondrial RNA targets and the inhibition of translation that results therefrom. This oligonucleotide-based inhibition of translation and ultimately gene expression is the result of one or more cellular mechanisms, which may include but is not limited to (i) direct (steric) blockage of translation, (ii) RNase H-mediated inhibition, and (iii) RNAi-mediated inhibition (e.g., short interfering-RNA (siRNA), microRNA (miRNA), Modulation of Splicing, Inhibition of noncoding RNA and single-stranded RNAi (ssRNAi)).

The history of antisense technology has revealed that while determination of antisense oligonucleotides that bind to mRNA is relatively straight forward, the optimization of antisense oligonucleotides that have true potential to inhibit gene expression and therefore be good clinical candidates is not. Being based on oligonucleotides, antisense technology has the inherent problem of being unstable in vivo and having the potential to produce off-target effects, for example unintended immune stimulation (Agrawal & Kandimalla (2004) Nature Biotech. 22:1533-1537).

Approaches to optimizing each of these technologies have focused on addressing biostability, affinity to RNA target, cell permeability, and in vivo activity. Often, these have represented competing considerations. For example, traditional antisense oligonucleotides utilized phosphodiester internucleotide linkages, which proved to be too biologically unstable to be effective. Thus, there was a focus on modifying antisense oligonucleotides to render them more biologically stable. Early approaches focused on modifying the inter-nucleotide linkages to make them more resistant to degradation by cellular nucleases. However, these modifications may cause the molecules to decrease their target specificity and produced unwanted biological activities.

Additionally, throughout oligonucleotide research, it has been recognized that these molecules are susceptible in vivo to degradation by exonucleases, with the primary degradation occurring from the 3′-end of the molecule (Temsamani et al. (1993) Analytical Bioc. 215:54-58). As such, approaches to avoid this exonuclease activity have utilized.

Despite considerable research, the efforts to improve the stability and maintain RNA target recognition, without off-target effects has not generally produced oligonucleotides that would be perceived having higher probability of clinical success. Accordingly, if an oligonucleotide-based approach to down-regulating gene expression is to be successful, there is still a need for optimized antisense oligonucleotides that most efficiently achieve this result. There are largely two key mechanisms of antisense activity. The first mechanism involves an antisense oligonucleotide hybridizing to a target RNA and the duplex formed activates RNase H, thereby cutting the targeted RNA and inhibiting the expression. The second mechanism is when an antisense oligonucleotide hybridizes to the target and blocks the processing of targeted RNA, including splicing, and thereby inhibiting or increasing the gene expression. This mechanism of antisense binding could also lead to nonsense mediated decay thereby inhibiting or increasing the gene expression. In use of both of these approaches, off-target effects have been observed and new design of antisense are needed to mitigate off target activity and increase potency.

For modulation of splicing, an antisense oligonucleotide is designed to bind to the targeted RNA with high affinity and selectivity. To date, antisense candidates employed for this mechanism includes modified RNA oligonucleotides such as 2′-O-methyl oligoribonucleoside, which were used in the very first study to modulate splicing in cells. (Sierakowska et al., (1996) Proc Natl Acad Sci USA, v93(23): 12840-4; Wilton et al., Neuromuscul Discord (1999) v9(5): 330-8). Since then, several other modified oligonucleotides have been evaluated, such as oligonucleotides having 2′-methoxyethoxy, LNA, HNA, CeNa, ANA or mixtures of these modifications.

However, other new designs are needed.

### SUMMARY OF THE INVENTION

The invention provides a method for modulating RNA processing comprising administering an antisense oligonucleotide comprising 14 to 30 linked nucleotides having at least 12 contiguous nucleobases complementary to an equal length portion of a target RNA, wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides, or combinations thereof.

The invention also provides a method for selecting a first mRNA transcript in a gene comprising at least two mRNA transcripts, the method comprising administering an antisense oligonucleotide comprising 14 to 30 linked nucleotides having at least 12 contiguous nucleobases complementary to an equal length portion of a target pre-mRNA; wherein the antisense oligonucleotide targets a splice site of the pre-mRNA for a second mRNA transcript thereby blocking the splice site for the second mRNA transcript and directing splicing of the pre-mRNA to the first mRNA transcript; and wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides or combinations thereof.

The invention also provides a method of treating a disease or disorder in a subject wherein modulating RNA processing would be beneficial to treat the subject, the method comprising administering an antisense oligonucleotide comprising 14 to 30 linked nucleotides having at least 12 contiguous nucleobases complementary to an equal length portion of a target RNA, wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides, or combinations thereof.

The invention also provides a method of inducing nonsense mediated decay of a target RNA comprising administering an antisense oligonucleotide comprising 14 to 30 linked nucleotides having at least 12 contiguous nucleobases complementary to an equal length portion of a target RNA, wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides, or combinations thereof.

The invention also provides a method of increasing a level of mRNA encoding a protein or a functional mRNA and increasing expression of the protein or the functional mRNA comprising administering an antisense oligonucleotide comprising 14 to 30 linked nucleotides having at least 12 contiguous nucleobases complementary to an equal length portion of a target RNA, wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides, or combinations thereof.

The invention also provides an antisense oligonucleotide comprising 14 to 30 linked nucleotides having at least 12 contiguous nucleobases complementary to an equal length portion of a target pre-mRNA comprising a retained intron, wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides, or combinations thereof.

### BRIEF DESCRIPTION OF THE DRAWINGS

FIG. 1A and FIG. 1B is a schematic of embodiments of the present invention.

### DETAILED DESCRIPTION

The present invention is directed to compounds, compositions, and methods useful for modulating gene splicing. In some embodiments, modulating gene splicing increases expression of a target protein, suppresses the expression of undesired protein or a target functional RNA.

By convention, sequences discussed herein are set forth 5′ to 3′ unless other specified. Moreover, a strand containing the sequence of a SEQ ID NO has that sequence from 5′ to 3′ unless otherwise specified.

The term “3′”, when used directionally, generally refers to a region or position in a polynucleotide or oligonucleotide 3′ (toward the 3′ end of the nucleotide) from another region or position in the same polynucleotide or oligonucleotide. The term “3′ end” generally refers to the 3′ terminal nucleotide of the component oligonucleotides.

The term “5′”, when used directionally, generally refers to a region or position in a polynucleotide or oligonucleotide 5′ (toward the 5′end of the nucleotide) from another region or position in the same polynucleotide or oligonucleotide. As used herein, the term “5′ end” generally refers to the 5′ terminal nucleotide of the component oligonucleotide.

The term “about” generally means that the exact number is not critical. Thus, oligonucleotides having one or two fewer nucleoside residues, or from one to several additional nucleoside residues are contemplated as equivalents of each of the embodiments described above.

“Antisense activity” means any detectable or measurable activity attributable to the hybridization of antisense oligonucleotide compound to its target nucleic acid. In certain embodiments, antisense activity is a decrease in the amount or expression of a target nucleic acid or protein encoded by such target nucleic acid. In certain embodiments, antisense activity is the modulation of splicing and thereby inhibiting or increasing the expression of protein encoded by such target nucleic acid.

“Antisense inhibition” means reduction of target nucleic acid levels or target protein levels in the presence of an antisense oligonucleotide complementary to a target nucleic acid as compared to target nucleic acid levels or target protein levels in the absence of the antisense oligonucleotide.

“Antisense oligonucleotide” means a single-stranded oligonucleotide having a nucleobase sequence that permits hybridization to a corresponding region or segment of a target nucleic acid.

The term “co-administration” or “co-administered” generally refers to the administration of at least two different substances. Co-administration refers to simultaneous administration, as well as temporally spaced order of up to several days apart, of at least two different substances in any order, either in a single dose or separate doses.

The term “in combination with” generally means administering an oligonucleotide-based compound according to the invention and another agent useful for treating a disease or condition that does not abolish the activity of the compound in the course of treating a patient. Such administration may be done in any order, including simultaneous administration, as well as temporally spaced order from a few seconds up to several days apart. Such combination treatment may also include more than a single administration of the compound according to the invention and/or independently the other agent. The administration of the compound according to the invention and the other agent may be by the same or different routes.

The term “individual” or “subject” or “patient” generally refers to a mammal, such as a human. The term “mammal” is expressly intended to include warm blooded, vertebrate animals, including, without limitation, humans, non-human primates, rats, mice, cats, dogs, horses, cattle, cows, pigs, sheep and rabbits. As used herein, “individual in need thereof” refers to a human or non-human animal selected for treatment or therapy that is in need of such treatment or therapy.

As used herein, “inhibiting the expression or activity” refers to a reduction or blockade of the expression or activity of an RNA or protein and does not necessarily indicate a total elimination of expression or activity.

The term “nucleoside” generally refers to compounds consisting of a sugar, usually ribose, deoxyribose, pentose, arabinose or hexose, and a purine or pyrimidine base. For purposes of the invention, a base is considered to be non-natural if it is not guanine, cytosine, adenine, thymine or uracil and a sugar is considered to be non-natural if it is not β-ribo-furanoside or 2′-deoxyribo-furanoside.

The term “nucleotide” generally refers to a nucleoside comprising a phosphorous-containing group attached to the sugar. As used herein, “linked nucleosides” may or may not be linked by phosphate linkages and thus includes, but is not limited to, “linked nucleotides.” As used herein, “linked nucleosides” are nucleosides that are connected in a continuous sequence (i.e., no additional nucleosides are present between those that are linked).

The term “nucleic acid” encompasses a genomic region or an RNA molecule transcribed therefrom. In some embodiments, the nucleic acid is mRNA. In some embodiments, the nucleic acid is microRNA. In some embodiments, the nucleic acid is ncRNA.

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified. As used herein, “nucleobase sequence” means the order of contiguous nucleobases independent of any sugar, linkage, or nucleobase modification.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “modified nucleoside” means a nucleoside comprising at least one chemical modification compared to naturally occurring RNA or DNA nucleosides. Modified nucleosides comprise a modified sugar moiety and/or a modified nucleobase.

As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA). In certain embodiments, an oligonucleotide comprises only unmodified ribonucleosides (RNA) and/or unmodified deoxyribonucleosides (DNA). In certain embodiments, an oligonucleotide comprises one or more modified nucleosides.

As used herein, “modified oligonucleotide” means an oligonucleotide comprising at least one modified nucleoside and/or at least one modified sugar.

As used herein “internucleoside linkage” means a covalent linkage between adjacent nucleosides in an oligonucleotide. As used herein “naturally occurring internucleoside linkage” means a 3′ to 5′ phosphodiester linkage. As used herein, “modified internucleoside linkage” means any internucleoside linkage other than a naturally occurring internucleoside linkage.

The phrase “an oligonucleotide that is complementary to a single-stranded RNA sequence” and the like, means that the oligonucleotide forms a sufficient number of hydrogen bonds through Watson-Crick interactions of its nucleobases with nucleobases of the single-stranded RNA sequence to form a double helix with the single-stranded RNA sequence under physiological conditions. This is in contrast to oligonucleotides that form a triple helix with a double-stranded DNA or RNA through Hoogsteen hydrogen bonding.

As used herein, “chemical modification” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

The term “complementary” is intended to mean an oligonucleotide that binds to the nucleic acid sequence under physiological conditions, for example, by Watson-Crick base pairing (interaction between oligonucleotide and single-stranded nucleic acid) or by Hoogsteen base pairing (interaction between oligonucleotide and double-stranded nucleic acid) or by any other means, including in the case of an oligonucleotide, binding to RNA and causing pseudoknot formation. Binding by Watson-Crick or Hoogsteen base pairing under physiological conditions is measured as a practical matter by observing interference with the function of the nucleic acid sequence.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Hybridization” means the annealing of complementary nucleic acid molecules. In certain embodiments, complementary nucleic acid molecules include an antisense compound and a target nucleic acid.

“Nonsense mediated decay” means any number of cellular mechanisms independent of RNase H or RISC that degrade mRNA or pre-mRNA. In certain embodiments, nonsense mediated decay eliminates and/or degrades mRNA transcripts that contain premature stop codons. In certain embodiments, nonsense mediated decay eliminates and/or degrades any form of aberrant mRNA and/or pre-mRNA transcripts.

The term “pharmaceutically acceptable” means a non-toxic material that does not interfere with the effectiveness of a compound according to the invention or the biological activity of a compound according to the invention.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

The term “prophylactically effective amount” generally refers to an amount sufficient to prevent or reduce the development of an undesired biological effect.

As used herein, “sugar moiety” means a naturally occurring sugar moiety or a modified sugar moiety of a nucleoside. As used herein, “naturally occurring sugar moiety” means a ribofuranosyl as found in naturally occurring RNA or a deoxyribofuranosyl as found in naturally occurring DNA. As used herein, “modified sugar moiety” means a substituted sugar moiety or a sugar surrogate, such as, but not limited, to 2′ modified sugars or constrained sugars.

The term “therapeutically effective amount” or “pharmaceutically effective amount” generally refers to an amount sufficient to affect a desired biological effect, such as a beneficial result, including, without limitation, prevention, diminution, amelioration or elimination of signs or symptoms of a disease or disorder. Thus, the total amount of each active component of the pharmaceutical composition or method is sufficient to show a meaningful patient benefit, for example, but not limited to, healing of chronic conditions characterized by immune stimulation. Thus, a “pharmaceutically effective amount” will depend upon the context in which it is being administered. A pharmaceutically effective amount may be administered in one or more prophylactic or therapeutic administrations. When applied to an individual active ingredient, administered alone, the term refers to that ingredient alone. When applied to a combination, the term refers to combined amounts of the active ingredients that result in the therapeutic effect, whether administered in combination, serially or simultaneously.

The term “treatment” generally refers to an approach intended to obtain a beneficial or desired result, which may include alleviation of symptoms, or delaying or ameliorating a disease progression.

The term “gene expression” generally refers to process by which information from a gene is used in the synthesis of a functional gene product, which may be a protein. The process may involve transcription, RNA splicing, translation, and post-translational modification of a protein, and may include mRNA, pre-mRNA, noncoding RNA, snoRNA, ribosomal RNA, and other templates for protein synthesis.

“Targeting” or “targeted” means the process of design and selection of an antisense oligonucleotide that will specifically hybridize to a target nucleic acid and induces a desired effect. “Target gene”, “target allele”, “target nucleic acid,” “target RNA,” “target mRNA,” and “target RNA transcript” all refer to a nucleic acid an antisense oligonucleotide that will specifically hybridize. A “target allele” is an allele whose expression is to be selectively targeted. “Target segment”, “target region”, and “target site” all refer to the sequence of nucleotides of a target nucleic acid to which antisense oligonucleotide is targeted.

A target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region.

Certain embodiments provide compositions and methods comprising administering to an animal an antisense compound or composition disclosed herein. In certain embodiments, administering the antisense compound prevents, treats, ameliorates, or slows progression of disease or condition related to the expression of a gene or activity of a protein. In certain embodiments, the animal is a human.

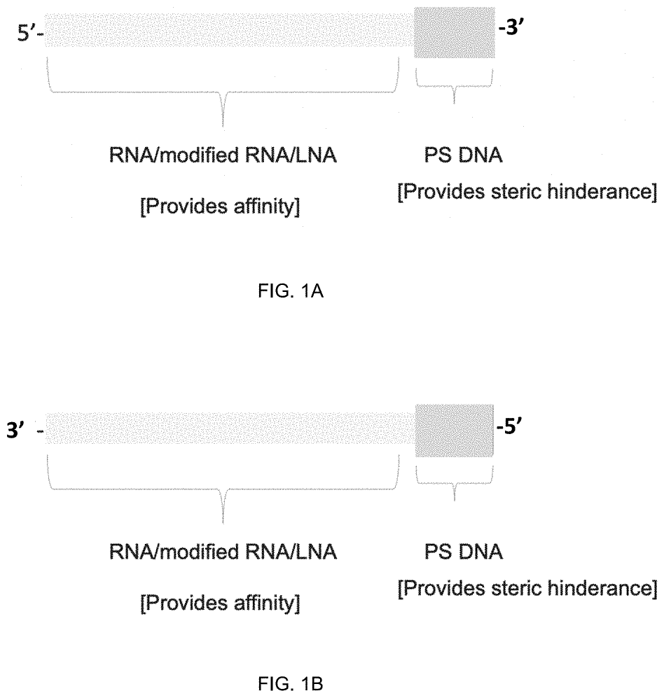

The present invention provides a new design of an antisense oligonucleotide for modulating splicing. In this design, the antisense oligonucleotide has two domains (see FIG. 1). The first domain is comprised of ribonucleotides (RNA), modified RNA or combinations thererof, which provide affinity to target RNA. The second domain comprised of phosphodiester or phosphorothioate oligodeoxynucleotide (DNA) which allows recruitment of RNase H but does not allow RNase H to cleave the antisense oligonucleotide-target RNA duplex. The recruitment of RNase H and its binding to the oligonucleotide-target RNA duplex, provides steric hinderance at the duplex site and promotes splicing. As used herein, modified RNA includes, but is not limited to, 2′-substituted, non-ionic or constrained sugar nucleotides.

Any of the methods disclosed herein comprises administering an antisense oligonucleotide as disclosed herein.

In some embodiments, the invention provides a method of modulating splicing. In some embodiments, the invention provides a method of modulating RNA splicing. In embodiments, the RNA includes, but is not limited to, pre-mRNA, mRNA, non-coding RNA. In embodiments, the RNA is pre-mRNA. In embodiments, the RNA is mRNA. In embodiments, the RNA is non-coding RNA. In some embodiments, the target RNA comprises a retained intron.

In some embodiments, the target pre-mRNA comprises a retained intron. In some embodiments, the retained intron is flanked on one or both sides by an exon. In some embodiments, an exon flanks the 5′ splice site of the retained intron. In some embodiments, an exon flanks the 3′ splice site of the retained intron. In some embodiments, an exon flanks the 5′ splice site of the retained intron and an exon flanks the 3′ splice site of the retained intron.

In some embodiments, the retained intron is constitutively spliced from the target RNA; thereby increasing a level of mRNA encoding a protein or a functional mRNA and increasing expression of the protein or the functional mRNA. In some embodiments, the invention provides a method of increasing a level of mRNA encoding a protein or a functional mRNA and increasing expression of the protein or the functional mRNA.

In some embodiments, the method of modulating splicing is useful to treat a subject having a condition caused by a deficient amount or activity of a protein or a deficient amount or activity of a functional mRNA; and wherein the deficient amount or activity of the protein or the functional mRNA is caused by haploinsufficiency of the target protein or the target functional RNA.

In some embodiments, the invention provides a method of treating a disease or disorder in a subject wherein modulating splicing would be beneficial to treat the subject. In embodiments, the disease or disorder is caused by a deficient amount or activity of a protein or a deficient amount or activity of a functional mRNA. In embodiments, the deficient amount or activity of the protein or the functional mRNA is caused by haploinsufficiency of the target protein or the target functional RNA.

In some embodiments, the antisense oligonucleotide compound comprises a sequence complementary to a region of the target RNA. In some embodiments, the antisense oligonucleotide compound comprises a sequence complementary to a region of the target RNA comprising a retained intron.

In one embodiment, the invention provides a method for selecting a first mRNA transcript in a gene comprising at least two mRNA transcripts, the method comprising administering an antisense oligonucleotide comprising 14 to 30 linked nucleotides having at least 12 contiguous nucleobases complementary to an equal length portion of a target pre-mRNA; wherein the antisense oligonucleotide targets a splice site of the pre-mRNA for a second mRNA transcript thereby blocking the splice site for the second mRNA transcript and directing splicing of the pre-mRNA to the first mRNA transcript; and wherein the antisense oligonucleotide comprises from 1 to 3 nucleotide regions comprising from 2 to 5 consecutive deoxyribonucleotides and the remaining nucleotides are 2′-substituted, non-ionic or constrained sugar nucleotides, or combinations thereof.

In any of embodiments herein, the retained intron is constitutively spliced from the target RNA; thereby increasing a level of mRNA encoding a protein or a functional mRNA and increasing expression of the protein or the functional mRNA. In some embodiments, the invention provides a method of increasing a level of mRNA encoding a protein or a functional mRNA and increasing expression of the protein or the functional mRNA.

In embodiments, the antisense oligonucleotide comprises 1 nucleotide region comprising from 2 to 5 consecutive deoxyribonucleotides and the remaining nucleotides are 2′-substituted, non-ionic or constrained sugar nucleotides, or combinations thereof.

In embodiments, the antisense oligonucleotide comprises 2 nucleotide regions comprising from 2 to 5 consecutive deoxyribonucleotides and the remaining nucleotides are 2′-substituted, non-ionic or constrained sugar nucleotides, or combinations thereof. In some embodiments, the 2 nucleotide regions are not contiguous.

In embodiments, the antisense oligonucleotide comprises 3 nucleotide regions comprising from 2 to 5 consecutive deoxyribonucleotides and the remaining nucleotides are 2′-substituted, non-ionic or constrained sugar nucleotides, or combinations thereof. In some embodiments, the deoxyribonucleotide regions are not contiguous.

In one embodiment, the invention provides a method for selecting a first mRNA transcript in a gene comprising at least two mRNA transcripts, the method comprising administering an antisense oligonucleotide comprising 14 to 30 linked nucleotides having at least 12 contiguous nucleobases complementary to an equal length portion of a target pre-mRNA; wherein the antisense oligonucleotide targets a splice site of the pre-mRNA for a second mRNA transcript thereby blocking the splice site for the second mRNA transcript and directing splicing of the pre-mRNA to the first mRNA transcript; and wherein the antisense oligonucleotide comprises from 1 to 3 nucleotide regions comprising from 2 to 4 consecutive deoxyribonucleotides and the remaining nucleotides are 2′-substituted, non-ionic or constrained sugar nucleotides, or combinations thereof.

In embodiments, the antisense oligonucleotide comprises 1 nucleotide region comprising from 2 to 4 consecutive deoxyribonucleotides and the remaining nucleotides are 2′-substituted, non-ionic or constrained sugar nucleotides, or combinations thereof.

In embodiments, the antisense oligonucleotide comprises 2 nucleotide regions comprising from 2 to 4 consecutive deoxyribonucleotides and the remaining nucleotides are 2′-substituted, non-ionic or constrained sugar nucleotides, or combinations thereof. In some embodiments, the 2 nucleotide regions are not contiguous.

In embodiments, the antisense oligonucleotide comprises 3 nucleotide regions comprising from 2 to 4 consecutive deoxyribonucleotides and the remaining nucleotides are 2′-substituted, non-ionic or constrained sugar nucleotides, or combinations thereof. In some embodiments, the deoxyribonucleotide regions are not contiguous.

In one embodiment, the invention provides a method for selecting a first mRNA transcript in a gene comprising at least two mRNA transcripts, the method comprising administering an antisense oligonucleotide comprising 14 to 30 linked nucleotides having at least 12 contiguous nucleobases complementary to an equal length portion of a target pre-mRNA; wherein the antisense oligonucleotide targets a splice site of the pre-mRNA for a second mRNA transcript thereby blocking the splice site for the second mRNA transcript and directing splicing of the pre-mRNA to the first mRNA transcript; and wherein the antisense oligonucleotide comprises a deoxyribonucleotide region comprising from 2 to 5 consecutive deoxyribonucleotides at the 3′ end of the antisense oligonucleotide and the remaining nucleotides are 2′-substituted, non-ionic or constrained sugar nucleotides, or combinations thereof. In embodiments, the deoxyribonucleotide region comprising from 4 consecutive deoxyribonucleotides at the 5′ end of the antisense oligonucleotide and the remaining nucleotides are 2′-substituted, non-ionic or constrained sugar nucleotides, or combinations thereof.

In one embodiment, the invention provides a method for selecting a first mRNA transcript in a gene comprising at least two mRNA transcripts, the method comprising administering an antisense oligonucleotide comprising 14 to 30 linked nucleotides having at least 12 contiguous nucleobases complementary to an equal length portion of a target pre-mRNA; wherein the antisense oligonucleotide targets a splice site of the pre-mRNA for a second mRNA transcript thereby blocking the splice site for the second mRNA transcript and directing splicing of the pre-mRNA to the first mRNA transcript; and wherein the antisense oligonucleotide comprises a deoxyribonucleotide region comprising from 2 to 5 consecutive deoxyribonucleotides at the 5′ end of the antisense oligonucleotide and the remaining nucleotides are 2′-substituted, non-ionic or constrained sugar nucleotides, or combinations thereof. In embodiments, deoxyribonucleotide region comprising from 4 consecutive deoxyribonucleotides at the 5′ end of the antisense oligonucleotide and the remaining nucleotides are 2′-substituted, non-ionic or constrained sugar nucleotides, or combinations thereof.

In certain embodiments, the invention provides a method of modulating processing of a target RNA comprising contacting a cell with an antisense oligonucleotide as describe herein, wherein the processing of the target precursor transcript is modulated. In some embodiments, processing of a target RNA includes, but is not limited to, splicing, cleavage, transport, translation, degradation of coding RNA and non coding RNA. In some embodiments, RNA processing includes inhibiting RNA binding proteins. In some embodiments, RNA processing comprises splicing of coding RNA and non coding RNA. In some embodiments, RNA processing comprises cleavage of coding RNA and non coding RNA. In some embodiments, RNA processing comprises transport of coding RNA and non coding RNA. In some embodiments, RNA processing comprises translation of coding RNA and non coding RNA. In some embodiments, RNA processing comprises degradation of coding RNA and non coding RNA.

In certain embodiments, a method of treating a disease or condition by modulating processing of a target precursor transcript, comprising administering an antisense oligonucleotide as described herein.

In certain embodiments, the invention provides a method of inducing nonsense mediated decay of a target RNA comprising administering an antisense oligonucleotide as described herein.

In certain embodiments, the antisense oligonucleotide described herein modulates splicing of one or more target nucleic acids and such modulation causes the degradation and/or reduction of the target nucleic acid through nonsense mediated decay.

In certain embodiments, an antisense oligonucleotide described herein complementary to a target nucleic acid may increase inclusion of an exon, the inclusion of which causes the nonsense mediated decay pathway to recognize and degrade the exon containing mRNA.

In certain embodiments, an antisense oligonucleotide described herein complementary to a target nucleic acid may increase exclusion of an exon, the exclusion of which causes the nonsense mediated decay pathway to recognize and degrade the mRNA without the exon.

Nonsense mediated decay is a type of surveillance pathway that serves to reduce errors in aberrant gene expression through the elimination and/or degradation of aberrant mRNA transcripts. In certain embodiments, the mechanism of nonsense mediated decay selectively degrades mRNAs that result from errors in pre-mRNA processing. For example, many pre-mRNA transcripts contain a number of exons and introns that may be alternatively spliced to produce any number of mRNA transcripts containing various combinations of exons. The mRNA transcripts are then translated into any number of protein isoforms. In certain embodiments, pre-mRNA is processed in such a way to include one or more exons, the inclusion of which produces an mRNA that encodes or would encode a non-functional protein or a mis-folded protein. In certain embodiments, pre-mRNA is processed in such a way to include one or more exons, the inclusion of which produces an mRNA that contains a premature termination codon. In certain such embodiments, the nonsense mediated decay mechanism recognizes the mRNA transcript containing the extra exon and degrades the mRNA transcript prior to translation. In certain such embodiments, the nonsense mediated decay mechanism recognizes the mRNA transcript containing the premature termination codon and degrades the mRNA transcript prior to translation.

In certain embodiments, pre-mRNA is processed in such a way to exclude one or more exons, the exclusion of which produces an mRNA that encodes a non-functional protein. In certain embodiments, pre-mRNA is processed in such a way to exclude one or more exons, the exclusion of which produces an mRNA that contains a premature termination codon. In certain such embodiments, the nonsense mediated decay mechanism recognizes the mRNA transcript missing the exon and degrades the mRNA transcript prior to translation. In certain such embodiments, the nonsense mediated decay mechanism recognizes the mRNA transcript missing the exon and containing the premature termination codon and degrades the mRNA transcript prior to translation.

Without wishing to be bound to any particular theory, the antisense oligonucleotide of the invention allows the antisense oligonucleotide to bind the target RNA and complex with RNase H; however, the antisense oligonucleotide becomes RNase H inactive. In other words, the antisense oligonucleotide/target RNA-RNase H complex will not be cleaved by RNase H. In some embodiments, the antisense oligonucleotide is administered locally.

In certain embodiments, antisense compounds comprise or consist of an oligonucleotide comprising a region that is complementary to a target nucleic acid. In certain embodiments, the target nucleic acid is an endogenous RNA molecule. In certain embodiments, the target nucleic acid is a pre-mRNA. In certain embodiments, an antisense oligonucleotide modulates splicing of a pre-mRNA.

In some embodiments, the antisense oligonucleotides are complementary to a nucleotide sequence of a target pre-mRNA, wherein the antisense oligonucleotides comprise 14 to 30 linked nucleotides having at least 12 contiguous nucleobases complementary to an equal length portion of a target pre-mRNA, wherein the antisense oligonucleotide comprises from 1 to 3 nucleotide regions comprising from 2 to 5 consecutive deoxyribonucleotides and the remaining nucleotides are 2′-substituted, non-ionic, or constrained sugar nucleotides, or combinations thereof.

In some embodiments, the antisense oligonucleotides are complementary to a nucleotide sequence of a target pre-mRNA, wherein the antisense oligonucleotides comprise 14 to 30 linked nucleotides having at least 12 contiguous nucleobases complementary to an equal length portion of a target pre-mRNA, wherein the antisense oligonucleotide comprises from 1 to 3 nucleotide regions comprising from 2 to 4 consecutive deoxyribonucleotides and the remaining nucleotides are 2′-substituted, non-ionic, or constrained sugar nucleotides, or combinations thereof.

In some embodiments, the antisense oligonucleotides are complementary to a nucleotide sequence of a target pre-mRNA, wherein the antisense oligonucleotides comprise 14 to 30 linked nucleotides having at least 12 contiguous nucleobases complementary to an equal length portion of a target pre-mRNA, wherein the antisense oligonucleotide comprises a deoxyribonucleotide region comprising from 2 to 5 consecutive deoxyribonucleotides at the 3′ end of the antisense oligonucleotide and the remaining nucleotides are 2′-substituted, non-ionic, or constrained sugar nucleotides, or combinations thereof.

In some embodiments, the antisense oligonucleotides are complementary to a nucleotide sequence of a target pre-mRNA, wherein the antisense oligonucleotides comprise 14 to 30 linked nucleotides having at least 12 contiguous nucleobases complementary to an equal length portion of a target pre-mRNA, wherein the antisense oligonucleotide comprises a deoxyribonucleotide region comprising from 2 to 5 consecutive deoxyribonucleotides at the 5′ end of the antisense oligonucleotide and the remaining nucleotides are 2′-substituted, non-ionic, or constrained sugar nucleotides, or combinations thereof.

In embodiments, the antisense oligonucleotide comprises 1 region comprising from 2 to 5 consecutive deoxyribonucleotides and the remaining nucleotides are 2′-substituted, non-ionic or constrained sugar nucleotides, or combinations thereof. In embodiments, the antisense oligonucleotide comprises 2 deoxyribonucleotide regions each region independently comprising from 2 to 5 consecutive deoxyribonucleotides and the remaining nucleotides are 2′-substituted, non-ionic or constrained sugar nucleotides, or combinations thereof. In embodiments, the antisense oligonucleotide comprises 3 deoxyribonucleotide regions each region independently comprising from 2 to 5 consecutive deoxyribonucleotides and the remaining nucleotides are 2′-substituted, non-ionic or constrained sugar nucleotides, or combinations thereof.

In embodiments, the deoxyribonucleotide region comprises from 2 to 5 consecutive deoxyribonucleotides and the remaining nucleotides are 2′-substituted, non-ionic or constrained sugar nucleotides, or combinations thereof. In embodiments, the deoxyribonucleotide region comprises from 2 to 4 consecutive deoxyribonucleotides and the remaining nucleotides are 2′-substituted, non-ionic or constrained sugar nucleotides, or combinations thereof. In embodiments, the deoxyribonucleotide region comprises 4 consecutive deoxyribonucleotides and the remaining nucleotides are 2′-substituted, non-ionic or constrained sugar nucleotides, or combinations thereof.

In some embodiments, the 2′-substituted nucleotides are selected from, but not limited to, 2′-O-methylribonucleotides, 2′-O-methoxy-ethyl (2′-MOE) ribonucleotides, 2′-halogen (e.g., fluoro) nucleotides and morpholino modified nucleic acids. In some embodiments, the constrained sugar nucleotides included bicyclic nucleosides. In some embodiments, the bicyclic nucleosides include locked nucleosides and bridged nucleosides. In some embodiments, the constrained sugar nucleotides are selected from, but not limited to, locked nucleic acids (LNA), peptide nucleic acid (PNA), anhydrohexitol nucleic acids (HNA), cyclohexenyl nucleic acids (CeNA), altritol nucleic acids (ANA), constrained MOE (cMOE), constrained ethyl (cEt), ethylene bridged nucleic acid (ENA), serinol nucleic acid (SNA), and twisted intercalating nucleic acids (TINA). In some embodiments, non-ionic includes but is not limited to methylphosphonate, phosphotriesters, and morpholino (PMO). In some embodiments, the nucleotides can be 2′-substituted and have a constrained sugar.

In some embodiments, the antisense oligonucleotide comprises 1 deoxyribonucleotide region comprising 2, 3, 4, or 5 consecutive deoxyribonucleotides.

In some embodiments, the antisense oligonucleotide comprises 1 deoxyribonucleotide region comprising 2, 3, or 4, consecutive deoxyribonucleotides. In some embodiments, the antisense oligonucleotide comprises 1 deoxyribonucleotide region comprising 2 consecutive deoxyribonucleotides. In some embodiments, the antisense oligonucleotide comprises 1 deoxyribonucleotide region comprising 3 consecutive deoxyribonucleotides. In some embodiments, the antisense oligonucleotide comprises 1 deoxyribonucleotide region comprising 4 consecutive deoxyribonucleotides. In some embodiments, the consecutive deoxyribonucleotides are at the 3′ end of the antisense oligonucleotide.

In some embodiments, the consecutive deoxyribonucleotides are at the 5′ end of the antisense oligonucleotide.

In some embodiments, the antisense oligonucleotide comprises 2 deoxyribonucleotide regions each region independently comprising 2, 3, or 4, consecutive deoxyribonucleotides. In some embodiments, the antisense oligonucleotide comprises 3 deoxyribonucleotide regions each region independently comprising 2, 3, or 4, consecutive deoxyribonucleotides.

In some embodiments, the consecutive deoxyribonucleotides are at the 5′ end of the antisense oligonucleotide, at the 3′ end of the antisense oligonucleotide, are flanked by the 2′-substituted, non-ionic, or constrained sugar oligonucleotides, or combinations thereof. In some embodiments, the consecutive deoxyribonucleotides are at the 5′ end of the antisense oligonucleotide. In some embodiments, the consecutive deoxyribonucleotides are at the 3′ end of the antisense oligonucleotide. In some embodiments, the consecutive deoxyribonucleotides are flanked by the 2′-substituted oligoribonucleotides.

In some embodiments, the consecutive deoxyribonucleotides are naturally occurring nucleotides. In some embodiments, the consecutive deoxyribonucleotides are unmodified. In some embodiments, one or more of the consecutive deoxyribonucleotides are modified.

The antisense oligonucleotides of the invention are pharmaceutically acceptable. The antisense oligonucleotides of the invention are injectable. In some embodiments, the target RNA may be an mRNA. Certain embodiments provide an antisense oligonucleotide wherein the antisense oligonucleotide is single-stranded.

In some embodiments, the invention provides an antisense oligonucleotide compound 17 nucleotides in length nucleotides in length comprising at least 12 contiguous nucleobases complementary to an equal length portion of a target sequence.

In some embodiments, the invention provides an antisense oligonucleotide compound 18 to 25 nucleotides in length nucleotides in length comprising at least 12 contiguous nucleobases complementary to an equal length portion of a target sequence. In some embodiments, the antisense oligonucleotide compound is 18 nucleotides in length. In some embodiments, the antisense oligonucleotide compound is 19 nucleotides in length. In some embodiments, the antisense oligonucleotide compound is 20 nucleotides in length. In some embodiments, the antisense oligonucleotide compound is 21 nucleotides in length. In some embodiments, the antisense oligonucleotide compound is 22 nucleotides in length. In some embodiments, the antisense oligonucleotide compound is 23 nucleotides in length. In some embodiments, the antisense oligonucleotide compound is 24 nucleotides in length. In some embodiments, the antisense oligonucleotide compound is 25 nucleotides in length.

In some embodiments, the invention provides an antisense oligonucleotide compound 20 nucleotides in length nucleotides in length comprising at least 12 contiguous nucleobases complementary to an equal length portion of a target sequence. In some embodiments, the antisense oligonucleotide comprises nucleotide regions comprising from 2 to 4 consecutive deoxyribonucleotides at the 3′ end of the antisense oligonucleotide and the remaining nucleotides are 2′-substituted, non-ionic or constrained sugar nucleotides, or combinations thereof.

In some embodiments, the antisense oligonucleotides of the invention may be at least 14 nucleotides in length, for example between 14 to 30 nucleotides in length. Thus, the antisense oligonucleotides of the invention may be 14, 15, 16, 17, 18, 19, 20, 21, 22, 23, 24, 25, 26, 27, 28, 29, or 30 nucleotides in length. In some embodiments, the antisense oligonucleotides of the invention may be between 14 to 25 nucleotides in length. In some embodiments, the antisense oligonucleotides of the invention may be between 17 to 22 nucleotides in length. In some embodiments, the antisense oligonucleotides of the invention may be between 19 to 28 nucleotides in length.

The antisense oligonucleotides of the invention may be 17, 18, 19, 20, 21, or 22 nucleotides in length. In some embodiments, the antisense oligonucleotides of the invention may be 17 nucleotides in length. The antisense oligonucleotides of the invention may be 18 nucleotides in length. The antisense oligonucleotides of the invention may be 19 nucleotides in length. The antisense oligonucleotides of the invention may be 20 nucleotides in length. The antisense oligonucleotides of the invention may be 21 nucleotides in length. The antisense oligonucleotides of the invention may be 22 nucleotides in length. The antisense oligonucleotides of the invention may be 23 nucleotides in length. The antisense oligonucleotides of the invention may be 24 nucleotides in length. The antisense oligonucleotides of the invention may be 25 nucleotides in length. The antisense oligonucleotides of the invention may be 26 nucleotides in length. The antisense oligonucleotides of the invention may be 27 nucleotides in length. The antisense oligonucleotides of the invention may be 28 nucleotides in length. The antisense oligonucleotides of the invention may be 29 nucleotides in length. The antisense oligonucleotides of the invention may be 30 nucleotides in length.

The natural or unmodified bases in RNA are adenine (A) and guanine (G), and the pyrimidine bases cytosine (C) and uracil (U) (DNA has thymine (T)). In contrast, modified bases, also referred to as heterocyclic base moieties, include other nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

In certain embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine. Further modified nucleobases include tricyclic pyrimidines such as phenoxazine cytidine ([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. In certain embodiments, the modified nucleobase is a 5-methylcytosine.

Representative modified sugars include carbocyclic or acyclic sugars, sugars having substituent groups at one or more of their 2′, 3′ or 4′ positions and sugars having substituents in place of one or more hydrogen atoms of the sugar. In certain embodiments, the sugar is modified by having a substituent group at the 2′ position. In additional embodiments, the sugar is modified by having a substituent group at the 3′ position. In other embodiments, the sugar is modified by having a substituent group at the 4′ position. It is also contemplated that a sugar may have a modification at more than one of those positions, or that an antisense oligonucleotide may have one or more nucleotides with a sugar modification at one position and also one or more nucleotides with a sugar modification at a different position.

Sugar modifications contemplated in an antisense oligonucleotide include, but are not limited to, a sugar substituent group selected from: OH; F; O-, S-, or N-alkyl; O-, S-, or N-alkenyl; O-, S or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In some embodiments, these groups may be chosen from: O(CH₂)ₓOCH₃, O((CH₂)ₓO)_{y}CH₃, O(CH₂)ₓNH₂, O(CH₂)ₓCH₃, O(CH₂)ₓONH₂, and O(CH₂)ₓON((CH₂)ₓCH₃)₂, where x and y are independently from 1 to 10.

In some embodiments, the modified sugar comprises a substituent group selected from the following: C₁ to C₁₀ lower alkyl, substituted lower alkyl, alkenyl, alkynyl, alkaryl, aralkyl, O-alkaryl or O-aralkyl, SH, SCH₃, Cl, Br, CN, OCN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an antisense oligonucleotide, or a group for improving the pharmacodynamic properties of an antisense oligonucleotide, and other substituents having similar properties. In one embodiment, the modification includes 2′-methoxyethoxy (2′-O—CH₂CH₂OCH₃, which is also known as 2′-O-(2-methoxyethyl) or 2′-MOE) (Martin et al., 1995), that is, an alkoxyalkoxy group. Another modification includes 2′-dimethylaminooxyethoxy, that is, a O(CH₂)₂ON(CH₃)₂ group, also known as 2′-DMAOE and 2′-dimethylaminoethoxyethoxy (also known in the art as 2′-O-dimethyl-amino-ethoxy-ethyl or 2′-DMAEOE), that is, 2′-O—CH₂—O—CH₂—N(CH₃)₂.

Additional sugar substituent groups include allyl (—CH₂—CH═CH₂), —O-allyl CH₂—CH═CH₂), methoxy (—O—CH₃), aminopropoxy (—OCH₂CH₂CH₂NH₂), and fluoro (F). Sugar substituent groups on the 2′ position (2′-) may be in the arabino (up) position or ribo (down) position. One 2′-arabino modification is 2′-F. Other similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics, for example, cyclobutyl moieties, in place of the pentofuranosyl sugar. Examples of U.S. patents that disclose the preparation of modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, which are herein incorporated by reference in its entirety.

Representative sugar substituent groups include groups described in U.S. Patent Application Publication 2005/0261218, which is hereby incorporated by reference. In particular embodiments, the sugar modification is a 2′-O-Me modification, a 2′ F modification, a 2′ H modification, a 2′ amino modification, a 4′ thioribose modification or a phosphorothioate modification on the carboxy group linked to the carbon at position 6′, or combinations thereof.

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety (also referred to as a constrained sugar). In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N3, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al, Bioorg. Med. Chem. Lett., 1998, 8, 2219-2222; Singh et al., J Org. Chem., 1998, 63, 10035-10039; Srivastava et al., J Am. Chem. Soc, 20017, 129, 8362-8379; Wengel et al., U.S. Pat. No. 7,053,207; Imanishi et al., U.S. Pat. No. 6,268,490; Imanishi et al., U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

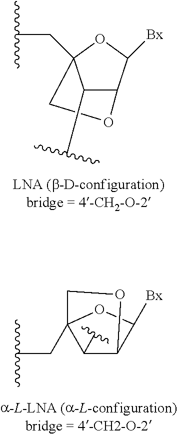

α-L-methyleneoxy (4′-CH2-0-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5 ‘-substituted and 4’-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

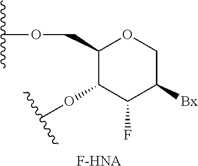

(“F-HNA”, see e.g., Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

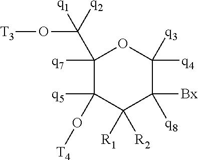

wherein, independently, for each of said modified THP nucleoside:

Bx is a nucleobase moiety;

T3 and T4 are each, independently, an internucleoside linking group linking the modified THP nucleoside to the remainder of an oligonucleotide or one of T3 and T4 is an internucleoside linking group linking the modified THP nucleoside to the remainder of an oligonucleotide and the other of T3 and T4 is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al, U.S. Pat. No. 5,185,444; and Summerton et al., U.S. Pat. No. 5,034,506). As used here, the term “morpholino” means a sugar surrogate having the following structure:

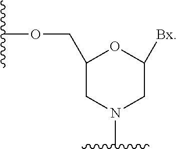

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

The nucleoside residues of the antisense oligonucleotides can be coupled to each other by any of the numerous known internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Methods of preparation of phosphorous-containing and non-phosphorous-containing internucleoside linkages are well known to those skilled in the art.

Such internucleoside linkages include, without limitation, phosphodiester, phosphorothioate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. In some embodiments, the synthetic antisense oligonucleotides of the invention may comprise combinations of internucleotide linkages. In some embodiments, the synthetic antisense oligonucleotides of the invention may comprise combinations of phosphorothioate and phosphodiester internucleotide linkages. In some embodiments more than half but less that all of the internucleotide linkages are phosphorothioate internucleotide linkages. In some embodiments all of the internucleotide linkages are phosphorothioate internucleotide linkages.

Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration.

In certain embodiments, the phosphorothioate linkages may be mixed Rp and Sp enantiomers, or they may be made stereoregular or substantially stereoregular in either Rp or Sp form. In embodiments where the linkages are mixed Rp and Sp enantiomers, the Rp and Sp forms may be at defined places within the antisense oligonucleotide or randomly placed throughout the oligonucleotide.

In certain embodiments, the invention provides antisense oligonucleotides as described herein and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N Y. Acad. Sci., 1992, 660, 306-309; Manoharan et al., Bioorg. Med. Chem. Lett, 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-0-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

The synthetic antisense compounds of the invention can be prepared by the art recognized methods such as phosphoramidite or H-phosphonate chemistry which can be carried out manually or by an automated synthesizer. The synthetic antisense compounds of the invention may also be modified in a number of ways without compromising their ability to hybridize to mRNA.

In some embodiments, the oligonucleotide-based compounds of the invention are synthesized by a linear synthesis approach.

At the end of the synthesis by either linear synthesis or parallel synthesis protocols, the oligonucleotide-based compounds of the invention may conveniently be deprotected with concentrated ammonia solution or as recommended by the phosphoramidite supplier, if a modified nucleoside is incorporated. The product oligonucleotide-based compounds are preferably purified by reversed phase HPLC, detritylated, desalted and dialyzed.

A non-limiting list of the antisense oligonucleotides of the invention are shown in Table 1. The antisense oligonucleotides in Table 1 are designed to induce exon 23 skipping in the mouse dystrophin gene transcript. Unless otherwise noted, the antisense oligonucleotides have phosphorothioate (PS) backbone linkages. Those skilled in the art will recognize, however, that other linkages, based on phosphodiester or non-phosphodiester moieties may be included.

TABLE 1

| | | SEQ |
|---|---|---|
| Compound # | Sequence | ID NO: |
| 1 | 5′-GGCCAAACCUCGGCUUACCU-3′ | 1 |
| 2 | 5′-GGCCAAACCUCGGCUUACCU-3′ | 2 |
| 3 | 5′-GGCCAAACCTCGGCUUACCU-3′ | 3 |
| 4 | 5′-GGCCAAACCUCGGCTUACCU-3′ | 4 |
| 5 | 5′-GGCCAAACCUCGGCUUACCU-3′ | 5 |
| 6 | 5′-GGCCAAACCUCGGCUUACCT-3′ | 6 |
| 7 | 5′-GGCCAAACCUCGGCUUACCU-3′ | 7 |
| 8 | 5′-GGCCAAACCUCGGCUUACCU-3′ | 8 |
| 9 | 5′-GGCCAAACCTCGGCUUACCU-3′ | 9 |
| 10 | 5′-GGCCAAACCUCGGCTTACCU-3′ | 10 |
| 11 | 5′-GGCCAAACCUCGGCUUACCT-3′ | 11 |
| 12 | 5′-GGCCAAACCUCGGCTTACCT-3′ | 12 |
| 13 | 5′-GGCCAAACCUCGGCTTACCT-3′ | 13 |
| 14 | 5′-GGCCAAACCUCGGCUTACCT-3′ | 14 |
| 15 | 5′-GGCCAAACCUCGGCUUACCT-3′ | 15 |
| 16 | 5′-GGCCAAACCUCGGCUUACCT-3′ | 16 |

underlined = deoxyribonucleotide;
non-underlined = 2′-O-methylnucleotide

In certain embodiments, the target nucleic acid is the murine sequence of the target. In certain embodiments, the target nucleic acid is the human sequence of the target.

The invention provides pharmaceutical compositions comprising the antisense oligonucleotides described herein and a pharmaceutically acceptable carrier. The term “carrier” generally encompasses any excipient, diluent, filler, salt, buffer, stabilizer, solubilizer, oil, lipid, lipid containing vesicle, microspheres, liposomal encapsulation, or other material for use in pharmaceutical formulations. It will be understood that the characteristics of the carrier, excipient or diluent will depend on the route of administration for a particular application. The preparation of pharmaceutically acceptable formulations containing these materials is described in, for example, Remington's Pharmaceutical Sciences, 18^{th} Edition, ed. A. Gennaro, Mack Publishing Co., Easton, Pa., 1990.

The composition may further comprise one or more other agents. Such agents may include but are not limited to, vaccines, antigens, antibodies, cytotoxic agents, chemotherapeutic agents (both traditional chemotherapy and modern targeted therapies), kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

The nucleic acid sequence to which an oligonucleotide according to the invention is complementary will vary, depending upon the agent to be inhibited. For example, the antisense oligonucleotides according to the invention can have an oligonucleotide sequence complementary to a cellular gene or gene transcript, the abnormal expression or product of which results in a disease state. The nucleic acid sequences of several such cellular genes have been described in the art. Antisense oligonucleotides according to the invention can have any oligonucleotide sequence so long as the sequence is partially or fully complementary to a target RNA nucleotide sequence.

In some embodiments, the antisense oligonucleotide may be at least 90% complementary over its entire length to a portion of the target RNA. In some embodiments, the antisense oligonucleotide may be at least 93% complementary over its entire length to a portion of the target RNA. In some embodiments, the antisense oligonucleotide may be at least 95% complementary over its entire length to a portion of the target RNA. In some embodiments, the antisense oligonucleotide may be at least 98% complementary over its entire length to a portion of the target RNA. In some embodiments, the antisense oligonucleotide may be at least 99% complementary over its entire length to a portion of the target RNA. In some embodiments, the antisense oligonucleotide may be 100% complementary over its entire length to a portion of the target RNA.

Certain embodiments provide a compound targeting a gene, wherein the compound comprises at least 8, at least 9, at least 10, at least 11, at least 12, at least 13, at least 14, at least 15, at least 16, at least 17, at least 18, at least 19, at least 20, at least 21, or 22 contiguous nucleobases complementary to an equal length portion of any target RNA. In some embodiments, the antisense oligonucleotide may comprise at least 12 contiguous nucleobases complementary to an equal length portion of the target RNA.

The antisense oligonucleotides of the invention may be administered alone or in combination with any other agent or therapy. Agents or therapies can be co-administered or administered concomitantly. Such agent or therapy may be useful for treating or preventing the disease or condition and does not diminish the gene expression modulation effect of the antisense oligonucleotide according to the invention. Agent(s) useful for treating or preventing the disease or condition includes, but is not limited to, vaccines, antigens, antibodies, preferably monoclonal antibodies, cytotoxic agents, kinase inhibitors, allergens, antibiotics, siRNA molecules, antisense oligonucleotides, TLR antagonist (e.g. antagonists of TLR3 and/or TLR7 and/or antagonists of TLR8 and/or antagonists of TLR9), chemotherapeutic agents (both traditional chemotherapy and modern targeted therapies), targeted therapeutic agents, activated cells, peptides, proteins, gene therapy vectors, peptide vaccines, protein vaccines, DNA vaccines, adjuvants, and co-stimulatory molecules (e.g. cytokines, chemokines, protein ligands, trans-activating factors, peptides or peptides comprising modified amino acids), or combinations thereof. Alternatively, the antisense oligonucleotides according to the invention can be administered in combination with other compounds (for example lipids or liposomes) to enhance the specificity or magnitude of the gene expression modulation of the antisense oligonucleotides according to the invention.

The antisense oligonucleotides of the invention may be administered can be by any suitable route, including, without limitation, parenteral, mucosal delivery, oral, sublingual, transdermal, topical, inhalation, intratumoral, intravenous, subcutaneous, intrathecal, intranasal, aerosol, intraocular, intratracheal, intrarectal, vaginal, by gene gun, dermal patch or in eye drop or mouthwash form. In any of the methods according to the invention, administration of antisense oligonucleotides according to the invention, alone or in combination with any other agent, can be directly to a tissue or organ such as, but not limited to, the bladder, liver, lung, kidney or lung. In certain embodiments, administration of antisense oligonucleotides according to the invention, alone or in combination with any other agent, is by intramuscular administration. In certain embodiments, administration of antisense oligonucleotides according to the invention, alone or in combination with any other agent, is by mucosal administration. In certain embodiments, administration of antisense oligonucleotides according to the invention, alone or in combination with any other agent, is by oral administration. In certain embodiments, administration of antisense oligonucleotides according to the invention, alone or in combination with any other agent, is by intrarectal administration. In certain embodiments, administration of antisense oligonucleotides according to the invention, alone or in combination with any other agent, is by intrathecal administration. In certain embodiments, administration of antisense oligonucleotides according to the invention, alone or in combination with any other agent, is by intratumoral administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Administration of the antisense oligonucleotides according to the invention can be carried out using known procedures using an effective amount and for periods of time effective to reduce symptoms or surrogate markers of the disease. For example, an effective amount of an antisense oligonucleotide according to the invention for treating a disease and/or disorder could be that amount necessary to alleviate or reduce the symptoms, or delay or ameliorate a tumor, cancer, or bacterial, viral or fungal infection. In the context of administering a composition that modulates gene expression, an effective amount of an antisense oligonucleotide according to the invention is an amount sufficient to achieve the desired modulation as compared to the gene expression in the absence of the antisense oligonucleotide according to the invention. The effective amount for any particular application can vary depending on such factors as the disease or condition being treated, the particular oligonucleotide being administered, the size of the subject, or the severity of the disease or condition. One of ordinary skill in the art can empirically determine the effective amount of a particular antisense oligonucleotide without necessitating undue experimentation.

When administered systemically, the therapeutic composition is preferably administered at a sufficient dosage to attain a blood level of compound according to the invention from about 0.0001 micromolar to about 10 micromolar. For localized administration, much lower concentrations than this may be effective, and much higher concentrations may be tolerated. Preferably, a total dosage of compound according to the invention ranges from about 0.001 mg per patient per day to about 200 mg per kg body weight per day. In certain embodiments, the total dosage may be 0.08, 0.16, 0.32, 0.48, 0.32, 0.64, 1, 10 or 30 mg/kg body weight administered daily, twice weekly or weekly. It may be desirable to administer simultaneously, or sequentially a therapeutically effective amount of one or more of the therapeutic compositions of the invention to an individual as a single treatment episode.

The methods according to this aspect of the invention are useful for model studies of gene expression. The methods are also useful for the prophylactic or therapeutic treatment of human or animal disease. For example, the methods are useful for pediatric and veterinary inhibition of gene expression applications.

Certain embodiments provide a kit for treating, preventing, or ameliorating a disease, disorder or condition as described herein wherein the kit comprises: (i) an antisense oligonucleotide as described herein; and optionally (ii) a second agent or therapy as described herein. A kit of the present invention can further include instructions for using the kit to treat, prevent, or ameliorate a disease, disorder or condition as described herein.

### Cell Culture and Antisense Compounds Treatment

The effects of antisense compounds on the level, activity or expression of target nucleic acids can be tested in vitro in a variety of cell types. Cell types used for such analyses are available from commercial vendors (e.g. American Type Culture Collection, Manassas, Va.; Zen-Bio, Inc., Research Triangle Park, N.C.; Clonetics Corporation, Walkersville, Md.) and are cultured according to the vendor's instructions using commercially available reagents (e.g. Invitrogen Life Technologies, Carlsbad, Calif.). Illustrative cell types include, but are not limited to, HepG2 cells, Hep3B cells, and primary hepatocytes.

### In Vitro Testing of Antisense Oligonucleotides

Described herein are methods for treatment of cells with antisense oligonucleotides, which can be modified appropriately for treatment with other antisense compounds.

Cells may be treated with antisense oligonucleotides when the cells reach approximately 60-80% confluency in culture.

One reagent commonly used to introduce antisense oligonucleotides into cultured cells includes the cationic lipid transfection reagent LIPOFECTIN (Invitrogen, Carlsbad, Calif.). Antisense oligonucleotides may be mixed with LIPOFECTIN in OPTI-MEM 1 (Invitrogen, Carlsbad, Calif.) to achieve the desired final concentration of antisense oligonucleotide and a LIPOFECTIN concentration that may range from 2 to 12 ug/mL per 100 nM antisense oligonucleotide.

Another reagent used to introduce antisense oligonucleotides into cultured cells includes LIPOFECTAMINE (Invitrogen, Carlsbad, Calif.). Antisense oligonucleotide is mixed with LIPOFECTAMINE in OPTI-MEM 1 reduced serum medium (Invitrogen, Carlsbad, Calif.) to achieve the desired concentration of antisense oligonucleotide and a LIPOFECTAMINE concentration that may range from 2 to 12 ug/mL per 100 nM antisense oligonucleotide.

Another technique used to introduce antisense oligonucleotides into cultured cells includes electroporation.

Cells are treated with antisense oligonucleotides by routine methods. Cells may be harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL Reagent (Invitrogen, Carlsbad, Calif.) according to the manufacturer's recommended protocols.

### Analysis of Inhibition of Target Levels or Expression

Inhibition of levels or expression of a target nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents may be obtained from Invitrogen (Carlsbad, Calif.). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc. Carlsbad, Calif.). Cyclophilin A expression is quantified by real time PCR, by being run simultaneously with the target, multiplexing, or separately. Total RNA is quantified using RIBOGREEN RNA quantification reagent (Invitrogen, Inc. Eugene, Oreg.). Methods of RNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a target nucleic acid. Methods for designing real-time PCR probes and primers are well known in the art and may include the use of software such as PRIMER EXPRESS Software (Applied Biosystems, Foster City, Calif.).

### Analysis of Protein Levels

Protein levels of can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art.

### In Vivo Testing of Antisense Compounds

Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. Calculation of antisense oligonucleotide dosage and dosing frequency is within the abilities of those skilled in the art and depends upon factors such as route of administration and animal body weight. Following a period of treatment with antisense oligonucleotides, RNA is isolated and changes in nucleic acid expression are measured.

### Certain Indications

In certain embodiments, provided herein are methods of treating an individual comprising administering one or more pharmaceutical compositions described herein. Certain embodiments include treating an individual in need thereof by administering to an individual a therapeutically effective amount of an antisense compound described herein.

In one embodiment, administration of a therapeutically effective amount of an antisense compound targeted to a nucleic acid is accompanied by monitoring of the corresponding target levels in an individual, to determine an individual's response to administration of the antisense compound. An individual's response to administration of the antisense compound may be used by a physician to determine the amount and duration of therapeutic intervention.

### EXAMPLES

### Synthesis of Antisense Oligonucleotides

Antisense oligonucleotides according to the invention can be synthesized by procedures that are well known in the art, such as phosphoramidate or H-phosphonate chemistry which can be carried out manually or by an automated synthesizer. For example, the antisense oligonucleotides of the invention may be synthesized by a linear synthesis approach.

ARNA compounds employed in the study have been synthesized using phosphoramidite chemistry. These protocols are described in detail, for example in https://pubs.rsc.org/en/content/chapter/bk9781788012096-00453/978-1-78801-209-6, which is incorporated herein by reference.

### Cell Culture and Transfection

H-2K^{b}-tsA58 mdx myoblasts 42,43 (H2K mdx cells) can be cultured and differentiated as described previously in the art. Briefly, when 60%-80% confluent myoblast cultures are treated with trypsin (Thermo Fisher Scientific) and seeded on 24-well plates pre-treated with 50 μg/mL poly-D-lysine (Merck Millipore), followed by 100 μg/ml Matrigel (Corning, supplied through In Vitro Technologies) at a density of 2×10⁴ cells/well. Cells can be differentiated into myotubes in DMEM (Thermo Fisher Scientific) containing 5% horse serum by incubating at 37° C. in 5% CO₂ for 24 hr. AOs can be complexed with Lipofectin (Thermo Fisher Scientific) at a ratio of 2:1 (w/w) (Lipofectin/AO) and used in a final transfection volume of 500 μL/well in a 24-well plate as per the manufacturer's instructions.

### RNA Extraction and RT-PCR

RNA can be extracted from transfected cells using Direct-zol RNA MiniPrep Plus with TRI Reagent (Zymo Research, supplied through Integrated Sciences) as per the manufacturer's instructions. The dystrophin transcripts can then be analyzed by RT-PCR using SuperScript III Reverse Transcriptase (Thermo Fisher Scientific) across exons 20-26. PCR products can be separated on 2% agarose gels in Tris-acetate-EDTA buffer, and the images captured on a Fusion Fx gel documentation system (Vilber Lourmat, Marne-la-Vallee, France). Densitometry can be performed by ImageJ software. The actual exon-skipping efficiency can be determined by expressing the amount of exon 23 skipped RT-PCR product as a percentage of total dystrophin transcript products. Results are shown in the following table.

| SEQ | | % of exon |
|---|---|---|
| ID NO: | Sequence | 23 skipping |
| 7 | 5′-GGCCAAACCUCGGCUUACCU-3′ | 34 |
| 8 | 5′-GGCCAAACCUCGGCUUACCU-3′ | 30 |
| 9 | 5′-GGCCAAACCTCGGCUUACCU-3′ | 0 |
| 10 | 5′-GGCCAAACCUCGGCTTACCU-3′ | 32 |
| 11 | 5′-GGCCAAACCUCGGCUUACCT-3′ | 42 |
| 12 | 5′-GGCCAAACCUCGGCTTACCT-3′ | 25 |
| 13 | 5′-GGCCAAACCUCGGCTTACCT-3′ | 25 |
| 14 | 5′-GGCCAAACCUCGGCUTACCT-3′ | 29 |
| 15 | 5′-GGCCAAACCUCGGCUUACCT-3′ | 34 |
| 16 | 5′-GGCCAAACCUCGGCUUACCT-3′ | 34 |

While this invention has been particularly shown and described with references to preferred embodiments thereof, it will be understood by those skilled in the art that various changes in form and details may be made therein without departing from the scope of the invention encompassed by the appended claims.

1. A method for modulating RNA processing comprising administering an antisense oligonucleotide comprising 14 to 30 linked nucleotides having at least 12 contiguous nucleobases complementary to an equal length portion of a target RNA, wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides, or combinations thereof.

2. A method for selecting a first mRNA transcript in a gene comprising at least two mRNA transcripts, the method comprising administering an antisense oligonucleotide comprising 14 to 30 linked nucleotides having at least 12 contiguous nucleobases complementary to an equal length portion of a target pre-mRNA; wherein the antisense oligonucleotide targets a splice site of the pre-mRNA for a second mRNA transcript thereby blocking the splice site for the second mRNA transcript and directing splicing of the pre-mRNA to the first mRNA transcript; and wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides or combinations thereof.

3. A method of treating a disease or disorder in a subject wherein modulating RNA processing would be beneficial to treat the subject, the method comprising administering an antisense oligonucleotide comprising 14 to 30 linked nucleotides having at least 12 contiguous nucleobases complementary to an equal length portion of a target RNA, wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides, or combinations thereof.

4. A method of inducing nonsense mediated decay of a target RNA comprising administering an antisense oligonucleotide comprising 14 to 30 linked nucleotides having at least 12 contiguous nucleobases complementary to an equal length portion of a target RNA, wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides, or combinations thereof.

5. A method of increasing a level of mRNA encoding a protein or a functional mRNA and increasing expression of the protein or the functional mRNA comprising administering an antisense oligonucleotide comprising 14 to 30 linked nucleotides having at least 12 contiguous nucleobases complementary to an equal length portion of a target RNA, wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides, or combinations thereof.

6. The method according to claim 5, wherein the target RNA comprises a retained intron.

7. The method according to claim 5, wherein the 2′-substituted nucleotides are selected from 2′ O-methylribonucleotides or 2′-MOE.

8. The method according to claim 5, wherein the antisense oligonucleotide comprises 1 region comprising from 2 to 5 consecutive deoxyribonucleotides.

9. The method according to claim 8, wherein the consecutive deoxyribonucleotides are at the 5′ end of the antisense oligonucleotide, at the 3′ end of the antisense oligonucleotide, flanked by at the 2′-substituted, non-ionic, or constrained sugar nucleotides, or combinations thereof.

10. The method according to claim 9, wherein the consecutive deoxyribonucleotides are at the 5′ end of the antisense oligonucleotide.

11. The method according to claim 9, wherein the consecutive deoxyribonucleotides are at the 3′ end of the antisense oligonucleotide.

12. The method according to claim 5, wherein the consecutive deoxyribonucleotides are 2-4 nucleotides in length.

13. The method according to claim 12, wherein the consecutive deoxyribonucleotides are 4 nucleotides in length.

14. The method according to claim 5, wherein an exon flanks the 5′ splice site of the retained intron.

15. The method according to claim 5, wherein an exon flanks the 3′ splice site of the retained intron.

16. The method according to claim 5, wherein an exon flanks the 5′ splice site of the retained intron and an exon flanks the 3′ splice site of the retained intron.

17. The method according to claim 2, wherein an exon flanks the 5′ side of the splice site for the second mRNA transcript.

18. The method according to claim 2, wherein an exon flanks the 3′ side of the splice site for the second mRNA transcript.

19. The method according to claim 2, wherein an exon flanks the 5′ side of the splice site for the second mRNA transcript and an exon flanks the 3′ side of the splice site for the second mRNA transcript.

20. The method according to claim 5, wherein the method is useful to treat a subject having a condition caused by a deficient amount or activity of a protein or a deficient amount or activity of functional mRNA expressed from the pre-mRNA.

21. The method according to claim 20, wherein the deficient amount or activity of target protein or the functional mRNA is caused by haploinsufficiency of the protein or the functional RNA.

22. The method according to claim 5, wherein the antisense oligonucleotide is part of a composition comprising a pharmaceutically acceptable carrier.

23. The method according to claim 5, wherein the antisense oligonucleotide is administered locally.

24. The method according to claim 5, wherein the antisense oligonucleotide comprises at least one phosphorothioate internucleotide linkage.

25. The method according to claim 24, wherein at least half of the internucleotide linkages are phosphorothioate.

26. The method according to claim 24, wherein all of the internucleotide linkages are phosphorothioate.

27. The method according to claim 5, wherein the antisense oligonucleotide is single stranded.

28. The method according to claim 5, wherein the antisense oligonucleotide is at least 90% complementary over its entire length to a portion of the target mRNA.

29. The method according to claim 5, wherein the RNA is selected from a pre-mRNA, mRNA, noncoding RNA.

30. An antisense oligonucleotide comprising 14 to 30 linked nucleotides having at least 12 contiguous nucleobases complementary to an equal length portion of a target pre-mRNA comprising a retained intron, wherein the antisense oligonucleotide comprises 1 to 3 regions each region independently comprising from 2 to 5 consecutive deoxyribonucleotides and the remaining nucleotides are 2′-substituted, non-ionic or constrained sugar nucleotides, or combinations thereof.

31. The oligonucleotide according to claim 30, wherein the 2′-substituted nucleotides are selected from 2′ O-methylribonucleotides or 2′-MOE.

32. The oligonucleotide according to claim 30, wherein the antisense oligonucleotide comprises 1 region comprising from 2 to 5 consecutive deoxyribonucleotides.

33. The oligonucleotide according to claim 32, wherein the consecutive deoxyribonucleotides are at the 5′ end of the antisense oligonucleotide, at the 3′ end of the antisense oligonucleotide, flanked by at the 2′-substituted, non-ionic, or constrained sugar nucleotides, or combinations thereof.

34. The oligonucleotide according to claim 33, wherein the consecutive deoxyribonucleotides are at the 5′ end of the antisense oligonucleotide.

35. The oligonucleotide according to claim 33, wherein the consecutive deoxyribonucleotides are at the 3′ end of the antisense oligonucleotide.

36. The oligonucleotide according to claim 30, wherein the consecutive deoxyribonucleotides are 2-4 nucleotides in length.

37. The oligonucleotide according to claim 36, wherein the consecutive deoxyribonucleotides are 4 nucleotides in length.

38. The oligonucleotide according to claim 30, wherein an exon flanks the 5′ splice site of the retained intron.

39. The oligonucleotide according to claim 30, wherein an exon flanks the 3′ splice site of the retained intron.

40. The oligonucleotide according to claim 30, wherein an exon flanks the 5′ splice site of the retained intron and an exon flanks the 3′ splice site of the retained intron.

41. The oligonucleotide according to claim 30, wherein the antisense oligonucleotide is administered locally.

42. The oligonucleotide according to claim 30, wherein the antisense oligonucleotide comprises at least one phosphorothioate internucleotide linkage.

43. The oligonucleotide according to claim 42, wherein at least half of the internucleotide linkages are phosphorothioate.

44. The oligonucleotide according to claim 42, wherein all of the internucleotide linkages are phosphorothioate.

45. The oligonucleotide according to claim 30, wherein the antisense oligonucleotide is single stranded.

46. The oligonucleotide according to claim 30, wherein the antisense oligonucleotide is at least 90% complementary over its entire length to a portion of the target mRNA.

47. The oligonucleotide according to claim 30, wherein the RNA is selected from a pre-mRNA, mRNA, and noncoding RNA.

48. A pharmaceutical composition comprising the oligonucleotide according to claim 30 and a pharmaceutically acceptable carrier.

49. The method according to claim 1, wherein processing of RNA comprises splicing.

50. The method according to claim 3, wherein processing of RNA comprises splicing.


SEQUENCE LISTING (16 sequences)

SEQ ID NO: 1 - DNA, 20 bp, Artificial Sequence
  2-substituted nucleotide
  modified_base (4)..(20): 2′-O-methylnucleotide
ggccaaaccucggcuuaccu

SEQ ID NO: 2 - DNA, 20 bp, Artificial Sequence
  2-substituted nucleotide
  modified_base (1)..(4): 2′-O-methylnucleotide
  modified_base (8)..(20): 2′-O-methylnucleotide
ggccaaaccucggcuuaccu

SEQ ID NO: 3 - DNA, 20 bp, Artificial Sequence
  2-substituted nucleotide
  modified_base (1)..(8): 2′-O-methylnucleotide
  modified_base (12)..(20): 2′-O-methylnucleotide
ggccaaacctcggcuuaccu

SEQ ID NO: 4 - DNA, 20 bp, Artificial Sequence
  2-substituted nucleotide
  modified_base (1)..(12): 2′-O-methylnucleotide
  modified_base (16)..(20): 2′-O-methylnucleotide
ggccaaaccucggctuaccu

SEQ ID NO: 5 - DNA, 20 bp, Artificial Sequence
  2-substituted nucleotide
  modified_base (4)..(16): 2′-O-methylnucleotide
  modified_base (19)..(20): 2′-O-methylnucleotide
ggccaaaccucggcuuaccu

SEQ ID NO: 6 - DNA, 20 bp, Artificial Sequence
  2-substituted nucleotide
  modified_base (1)..(17): 2′-O-methylnucleotide
ggccaaaccucggcuuacct

SEQ ID NO: 7 - DNA, 20 bp, Artificial Sequence
  2-substituted nucleotide
  modified_base (5)..(20): 2′-O-methylnucleotide
ggccaaaccucggcuuaccu

SEQ ID NO: 8 - DNA, 20 bp, Artificial Sequence
  2-substituted nucleotide
  modified_base (1)..(4): 2′-O-methylnucleotide
  modified_base (9)..(20): 2′-O-methylnucleotide
ggccaaaccucggcuuaccu

SEQ ID NO: 9 - DNA, 20 bp, Artificial Sequence
  2-substituted nucleotide
  modified_base (1)..(8): 2′-O-methylnucleotide
  modified_base (13)..(20): 2′-O-methylnucleotide
ggccaaacctcggcuuaccu

SEQ ID NO: 10 - DNA, 20 bp, Artificial Sequence
  2-substituted nucleotide
  modified_base (1)..(12): 2′-O-methylnucleotide
  modified_base (17)..(20): 2′-O-methylnucleotide
ggccaaaccucggcttaccu

SEQ ID NO: 11 - DNA, 20 bp, Artificial Sequence
  2-substituted nucleotide
  modified_base (1)..(16): 2′-O-methylnucleotide
ggccaaaccucggcuuacct

SEQ ID NO: 12 - DNA, 20 bp, Artificial Sequence
  2-substituted nucleotide
  modified_base (1)..(13): 2′-O-methylnucleotide
ggccaaaccucggcttacct

SEQ ID NO: 13 - DNA, 20 bp, Artificial Sequence
  2-substituted nucleotide
  modified_base (1)..(14): 2′-O-methylnucleotide
ggccaaaccucggcttacct

SEQ ID NO: 14 - DNA, 20 bp, Artificial Sequence
  2-substituted nucleotide
  modified_base (1)..(15): 2′-O-methylnucleotide
ggccaaaccucggcutacct

SEQ ID NO: 15 - DNA, 20 bp, Artificial Sequence
  2-substituted nucleotide
  modified_base (1)..(17): 2′-O-methylnucleotide
ggccaaaccucggcuuacct

SEQ ID NO: 16 - DNA, 20 bp, Artificial Sequence
  2-substituted nucleotide
  modified_base (1)..(18): 2′-O-methylnucleotide
ggccaaaccucggcuuacct